(19) United States
(12) Patent Application Publication
(10) Pub. No.: US 2025/0248853 A1
(43) Pub. Date: Aug. 7, 2025

(54) Wearable Noise-Reducing Device

(71) Applicant: Erin Raeman, Mint Hill, NC (US)

(72) Inventor: Erin Raeman, Mint Hill, NC (US)

(21) Appl. No.: 19/034,819

(22) Filed: Jan. 23, 2025

Related U.S. Application Data
(60) Provisional application No. 63/549,185, filed on Feb. 2, 2024.

(51) Int. Cl. A61F 11/12

(52) U.S. Cl. CPC A61F 11/12; A61F 2210/009

(57) ABSTRACT

A wearable noise-reducing device is provided. The device may have ear plugs which may be engaged to the user's ears for sound blocking and reducing purposes, and the device may be configured to be worn on the body of a user when not engaged to the user's ears. The device may include a first connector that may be coupled to a first ear plug, and the first ear plug may surround at least a portion of the first connector. A second connector may be coupled to a second ear plug, and the second ear plug may surround at least a portion of the second connector. The first connector and second connector may be configured to be removably coupled together. A flexible coupling, having an elongated length, may be coupled to both the first ear plug and to the second ear plug.

### CROSS REFERENCE TO RELATED APPLICATIONS

This application claims priority to and the benefit of the filing date of U.S. Provisional Application No. 63/549,185, filed on Feb. 2, 2024, entitled “Wearable Noise-Reducing Device”, which is hereby incorporated by reference in its entirety.

### FIELD OF THE INVENTION

This patent specification relates to the field of hearing protection devices. More specifically, this patent specification relates to a wearable noise-reducing device preferably having interchangeable ear plugs.

### BACKGROUND

People may need hearing protection when exposed to loud noises and environments to prevent potential hearing damage or loss. Prolonged exposure to loud sounds, whether in the workplace or recreational settings, can lead to noise-induced hearing loss (NIHL). Hearing protection is particularly important in industries such as construction, manufacturing, and entertainment where loud machinery, equipment, or music is common. Additionally, people can be exposed to potentially damaging noises in social and everyday situations, such as at weddings, parties, sporting events, and even during childcare.

Hearing protection devices come in various forms, including earplugs and earmuffs, and are designed to reduce the intensity of sound reaching the ears. Unfortunately, existing earplug devices and earmuff devices can be difficult to manage and are often not used as soon or as often are desired. Existing earplug devices can become easily lost or separated, and existing earplug devices having a corded connection inevitably become tangled. Existing earmuff devices are large and bulky which makes them difficult to store and wear.

Therefore, a need exists for novel hearing protection devices. A further need exists for novel hearing protection devices which do not suffer from the above-mentioned drawbacks of existing hearing protection devices.

### BRIEF SUMMARY OF THE INVENTION

A wearable noise-reducing device is provided. The device may include ear plugs which may be engaged to the user's ears for sound blocking and reducing purposes, and the device may be configured to be worn on the body of a user when not engaged to the user's ears.

In some embodiments, the device may include a first connector that may be coupled to a first ear plug, and the first ear plug may surround at least a portion of the first connector. A second connector may be coupled to a second ear plug, and the second ear plug may surround at least a portion of the second connector. The first connector and second connector may be configured to be removably coupled together. A flexible coupling, having an elongated length, may be coupled to both the first ear plug and to the second ear plug.

In some embodiments, the device may include a first connector that may be coupled to a first plug base. A first ear plug may be coupled to the first plug base, and the first ear plug may surround at least a portion of the first connector. A second connector may be coupled to a second plug base. A second ear plug may be coupled to the second plug base, and the second ear plug may surround at least a portion of the second connector. The first connector and second connector may be configured to be removably coupled together. A flexible coupling having an elongated length may be coupled to both the first plug base and to the second plug base.

Preferably, when not being worn for noise-reducing purposes, the device may be worn as a necklace with the flexible coupling extending around the majority of the user's neck and with the connectors coupled together. Preferably, to use the device for noise-reducing purposes, the connectors may be uncoupled from each other and the ear plugs may be inserted into the user's ears so that the ear plugs may protect the user's ears from undesired noise and sounds while the flexible coupling may maintain the connection between the ear plugs.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 depicts a perspective view of an example of a wearable noise-reducing device according to various embodiments described herein.

FIG. 2 illustrates a sectional, through line 2-2 shown in FIG. 1, elevation view of an example of a wearable noise-reducing device according to various embodiments described herein.

FIG. 3 shows a perspective, exploded view of an example of a first plug base and some example components that may be coupled to the first plug base according to various embodiments described herein.

FIG. 4 depicts a perspective, exploded view of an example of a second plug base and some example components that may be coupled to the second plug base according to various embodiments described herein.

FIG. 5 illustrates a sectional, through line 5-5 shown in FIG. 1, elevation view of an example of a wearable noise-reducing device according to various embodiments described herein.

FIG. 6 shows a sectional, through line 6-6 shown in FIG. 1, elevation view of an example of a wearable noise-reducing device according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

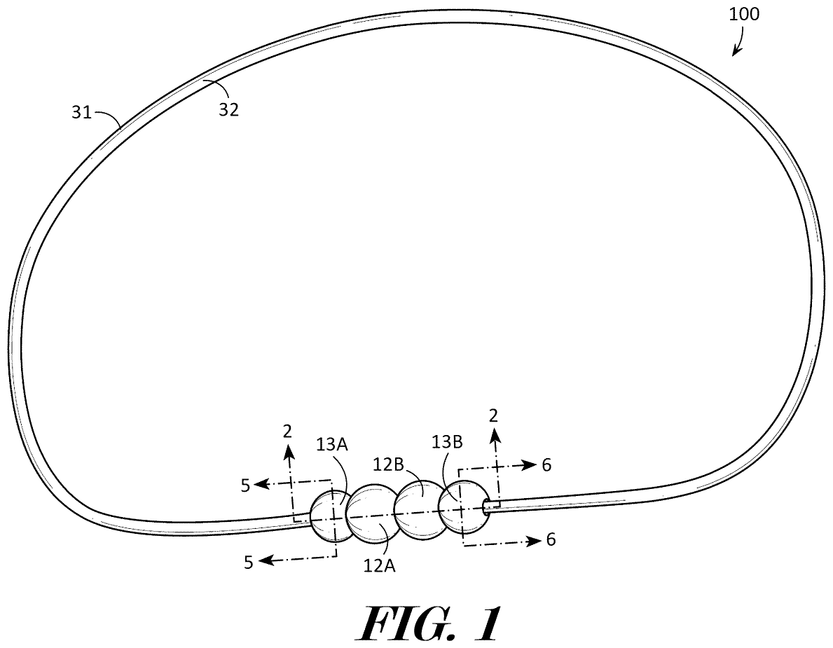

For purposes of description herein, the terms “upper,” “lower,” “left,” “right,” “rear,” “front,” “side,” “vertical,” “horizontal,” and derivatives thereof shall relate to the invention as oriented in FIG. 1. However, one will understand that the invention may assume various alternative orientations and step sequences, except where expressly specified to the contrary. Therefore, the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Although the terms “first,” “second,” etc. are used herein to describe various elements, these elements should not be limited by these terms. These terms are only used to distinguish one element from another element. For example, the first element may be designated as the second element, and the second element may be likewise designated as the first element without departing from the scope of the invention.

As used in this application, the term “about” or “approximately” refers to a range of values within plus or minus 20% of the specified number. Additionally, as used in this application, the term “substantially” means that the actual value is within about 10% of the actual desired value, more preferably within about 5% of the actual desired value and even more preferably within about 1% of the actual desired value of any variable, element or limit set forth herein.

A new wearable noise-reducing device is discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

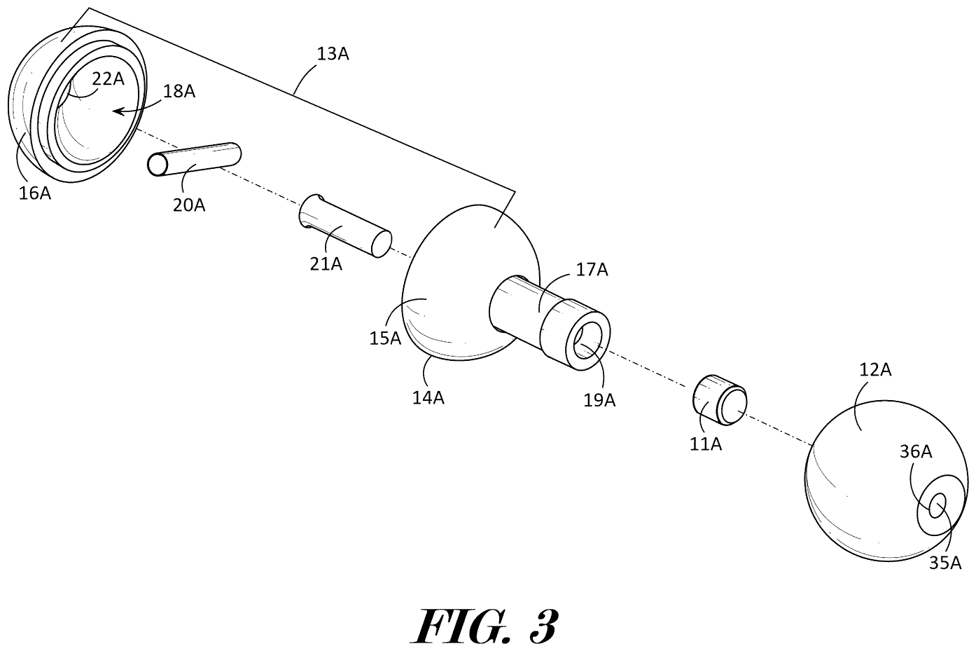

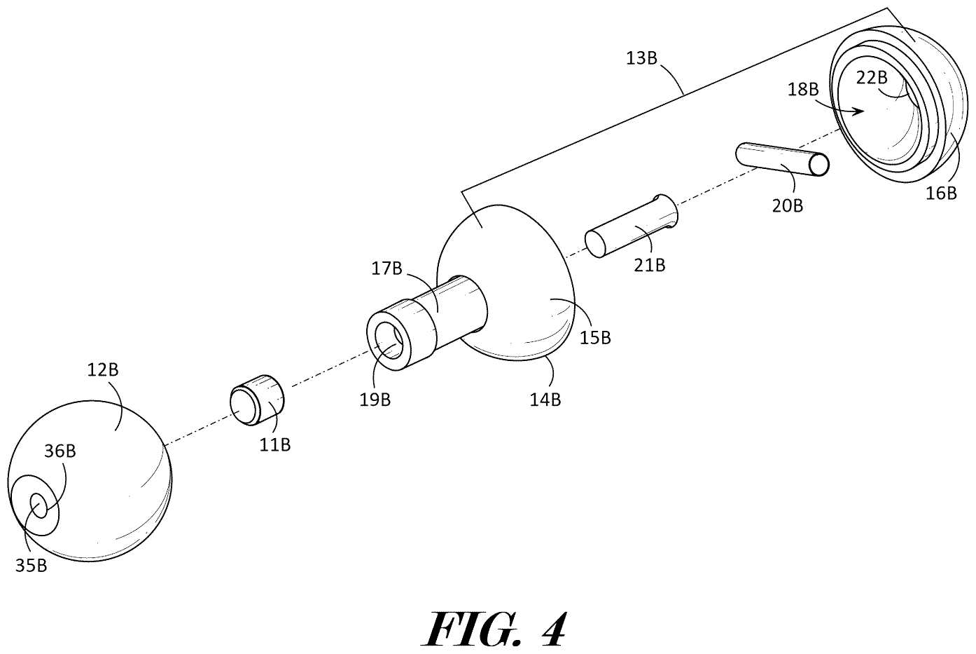

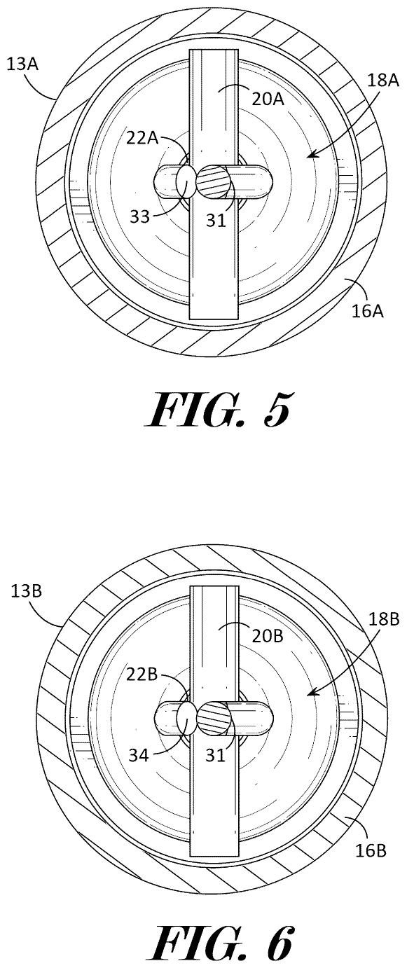

The present invention will now be described by example and through referencing the appended figures representing preferred and alternative embodiments. FIGS. 1-6 illustrate example components of a wearable noise-reducing device (“the device”) 100 according to various embodiments.

In some embodiments, the device 100 may comprise a first connector 11A that may be coupled to a first ear plug 12A, and the first ear plug 12A may surround at least a portion of the first connector 11A. A second connector 11B may be coupled to a second ear plug 12B, and the second ear plug 12B may surround at least a portion of the second connector 11B. The first connector 11A and second connector 11B may be configured to be removably coupled together. A flexible coupling 31 having an elongated length 32 may be coupled to both the first ear plug 12A and to the second ear plug 12B.

In some embodiments, the device 100 may comprise a first connector 11A that may be coupled to a first plug base 13A. A first ear plug 12A may be coupled to the first plug base 13A, and the first ear plug 12A may surround at least a portion of the first connector 11A. A second connector 11B may be coupled to a second plug base 13B. A second ear plug 12B may be coupled to the second plug base 13B, and the second ear plug 12A may surround at least a portion of the second connector 11B. The first connector 11A and second connector 11B may be configured to be removably coupled together. A flexible coupling 31 having an elongated length 32 may be coupled to both the first plug base 13A and to the second plug base 13B.

Preferably, when not being worn for noise-reducing purposes, the device 100 may be worn as a necklace with the flexible coupling 31 extending around the majority of the user's neck and with the connectors 11A, 11B, coupled together. Preferably, to use the device 100 for noise-reducing purposes, the connectors 11A, 11B, may be uncoupled from each other and the ear plugs 12A, 12B, may be inserted into the user's ears so that the ear plugs 12A, 12B, may protect the user's ears from undesired noise and sounds while the flexible coupling 31 may maintain the connection between the ear plugs 12A, 12B.

In some embodiments, the device 100 may comprise a first plug base 13A and a second plug base 13B that may be coupled together via a flexible coupling 31, and the plug bases 13A, 13B, may each be coupled to an ear plug 12A, 12B. A plug base 13A, 13B, may be configured in any size and shape. In preferred embodiments, a plug base 13A, 13B, may comprise a plug stop 14A, 14B, which may be sized and shaped to prevent over insertion of portions of the device 100, such as the ear plugs 12A, 12B, into a user's ear. Preferably, a plug stop 14A, 14B, may be configured with a spherical shape. In further embodiments, a stop 14A, 14B, may be configured in any size and shape. Preferably, a plug stop 14A, 14B, may generally comprise a spherical shape, and may be sized approximately the same size as ear plugs 12A, 12B, or may be sized to be larger than the ear plugs 12A, 12B.

In some embodiments, a plug base 13A, 13B, may comprise one or more base subunits 15A, 15B, 16A, 16B. For example, a first plug stop 14A of a first plug base 13A may comprise a first subunit 15A and a second subunit 16A, and a second plug stop 14B of a second plug base 13B may comprise a first subunit 15B and a second subunit 16B. The subunits 15A, 16A, of the first plug base 13A may be coupled together, and the subunits 15B, 16B, of the second plug base 13B may be coupled together.

In some embodiments, a first plug base 13A may comprise a first base projection 17A, and the first ear plug 12A and the first connector 11A may be coupled to the first base projection 17A. In some embodiments, a second plug base 13B may comprise a second base projection 17B, and the second ear plug 12B and the second connector 11B may be coupled to the second base projection 17B. A base projection 17A, 17B, may be configured in any shape and size. In preferred embodiments, a base projection 17A, 17B, may comprise an elongated shape that may extend away from a subunit 15A, 15B, of a plug stop 14A, 14B, that it may be coupled to. Preferably, a base projection 17A, 17B, may be configured in a generally cylindrical shape.

In some embodiments, a first plug base 13A may comprise a first base cavity 18A that may be disposed within the first plug stop 14A. In some embodiments, a second plug base 13B may comprise a second base cavity 18B that may be disposed within the second plug stop 14B. A base cavity 18A, 18B, may be configured in any size and shape, such as a generally spherical shape.

In some embodiments, a first base projection 17A may comprise a first base channel 19A, and/or a second base projection 17B may comprise a second base channel 19B. A base channel 19A, 19B, may comprise a recess, depression, conduit, or other opening of any size and shape, such as a generally cylindrical shape, in a base projection 17A, 17B. Optionally, a base channel 19A, 19B, may extend fully or partially through a base projection 17A, 17B. In preferred embodiments, a first base projection 17A may comprise a first base channel 19A that may extend through the first base projection 17A and into communication with the first base cavity 18A. In some embodiments, a second base projection 17B may comprise a second base channel 19B that may extend through the second base projection 17B and into communication with the second base cavity 18B.

In preferred embodiments, a base projection 17A, 17B, may comprise an acoustic dampener 21A, 21B. Preferably, an acoustic dampener 21A, 21B, may comprise a sound dampening or sound deadening material. In preferred embodiments, an acoustic dampener 21A, 21B, may comprise acoustic foam that may be an open celled foam used for acoustic treatment, such as may be made from polyurethane (either polyether or polyester), extruded melamine foam, etc. In further embodiments, an acoustic dampener 21A, 21B, may comprise silicone, rubber, acoustic mineral wool, echo absorber acoustic cotton, other types of foam materials, or any other material that may possess sound dampening or sound deadening properties. An acoustic dampener 21A, 21B, may be configured in any size and shape. Preferably, an acoustic dampener 21A, 21B, may comprise a generally cylindrical shape.

In preferred embodiments, a base projection 17A, 17B, may comprise an acoustic dampener 21A, 21, that may be disposed within the base channel 19A, 19B. In further preferred embodiments, a base projection 17A, 17B, may comprise an acoustic dampener 21A, 21, and a base channel 19A, 19B, that may extend fully through the base projection 17A, 17B, and into communication with a base cavity 18A, 18B, and the acoustic dampener 21A, 21, may be disposed within the base channel 19A, 19B, to acoustically isolate a connector 11A, 11B, coupled to the base projection 17A, 17B, from the base cavity 18A, 18B.

The device 100 may comprise a flexible coupling 31 which may couple a first plug base 13A and a second plug base 13B together even when the connectors 11A, 11B, are not coupled together. A flexible coupling 31 may comprise a first end 33 that may be coupled to a first plug base 13A and a second end 34 that may be coupled to a second plug base 13B. A flexible coupling 31 may comprise an elongated length 32 that may be made from or may comprise a flexible material. In preferred embodiments, a flexible coupling 31 may comprise an elongated length 32 of a flexible thermoplastic elastomer (TPE) material. In some embodiments, a flexible coupling 31 may comprise an elongated length 32 of a flexible silicone material. In preferred embodiments, a flexible coupling 31 may comprise an elongated length 32 of gold, silver, or other jewelry chain. In further embodiments, a flexible coupling 31 may comprise an elongated length 32 of silicone rope, fabric rope, beaded necklace material, or any other decorative or utilitarian elongated flexible material. In further embodiments, a flexible coupling 31 may comprise an elongated length 32 of a sound dampening material. Generally, a flexible coupling 31 may be available in a variety of colors and materials to accommodate consumer style preferences. Optionally, a flexible coupling 31 may be interchangeably coupled or removably coupled to the plug bases 13A, 13B.

A flexible coupling 31 may be coupled to the plug bases 13A, 13B, with any suitable coupling method. Preferably, each end 33, 34, of the flexible coupling 31 may be coupled to a plug base 13A, 13B, with each end 33, 34, positioned within a base cavity 18A, 18B. In some embodiments, a flexible coupling 31 may be coupled to a coupling retainer 20A, 20B, that may be positioned within a base cavity 18A, 18B, of the plug bases 13A, 13B, and the ends 33, 34, of the flexible coupling 31 may enter the base cavities 18A, 18B, via a base aperture 22A, 22B, that may be formed in each plug base 13A, 13B. Each end 33, 34, of the flexible coupling 31 may be coupled to a coupling retainer 20A, 20B, via heat bonding, adhesive, mechanical crimping, or any other suitable coupling method. A coupling retainer 20A, 20B, may comprise a unit of material, such as plastic, metal, etc., which may resist deformation and which may be sized and shaped to be positioned in a base cavity 18A, 18B, while being sized and shaped so as to be prevented from being pulled through a base aperture 22A, 22B, and out of the base cavity 18A, 18B. As an example, a coupling retainer 20A, 20B, may comprise a rod shape, ball shape or any other shape that may be prevented from passing through a base aperture 22A, 22B. Optionally, the ends 33, 34, of a flexible coupling 31 may be formed into coupling retainers 20A, 20B, such as by deforming, e.g., melting or heat deformation, the ends 33, 34, of a flexible coupling 31 into a size and shape that may be prevented from passing through a base aperture 22A, 22B. Optionally, a coupling retainer 20A, 20B, may comprise a first material which may resist deformation, such as a hard plastic, and the first material may be surrounded by a sound dampening material. For example, the first material may be wrapped in a sound dampening material and/or a vibration reducing material (e.g., heat shrink polymer, over molded with TPE, wrapping the first material in an acoustic dampening foam, etc.).

The device 100 may comprise two or more ear plugs 12A, 12B, that may be coupled to the plug bases 13A, 13B. Preferably, ear plugs 12A, 12B, may be made from or may comprise flexible materials, such as silicone, foam, or any other flexible material suitable for being inserted into or otherwise positioned in contact with a user's ear. Optionally, ear plugs 12A, 12B, may be made from or may comprise a flexible material that may be moldable or formed to fit a specific user's ear. Generally, ear plugs 12A, 12B, may be available in a variety of colors and materials to accommodate consumer style preferences. Preferably, the device 100 may comprise two or more ear plugs 12A, 12B, that may be configured in different shapes and sizes, such as spherical, ovoid, etc., so that the user may select ear plugs 12A, 12B, having a desired shape and size that is complementary to the user's ear. For example, the device 100 may comprise two or more ear plugs 12A, 12B, that are interchangeable and are available in different sizes (XS, S, M, L, XL) and may be available in different sound attenuation levels (e.g., 1 dB to 100 dB sound reduction).

In preferred embodiments, the device 100 may comprise two or more ear plugs 12A, 12B, that may be removably coupled to the plug bases 13A, 13B, so that the ear plugs 12A, 12B, may be interchangeable and selectable based on user preference. In other embodiments, an ear plug 12A, 12B, may be configured in any other shape and size that may be suitable for insertion into the ear, such as into the ear canal, of a user. In preferred embodiments, an ear plug 12A, 12B, may comprise an ear plug channel 35A, 35B, that may extend through the ear plug 12A, 12B. Optionally, an ear plug 12A, 12B, may comprise an ear plug aperture 36A, 36B, that the ear plug channel 35A, 35B, may be in communication with. Generally, portions of a plug base 13A, 13B, such as a base projection 17A, 17B, may be received in the ear plug channel 35A, 35B, such as by being frictionally fit and retained therein.

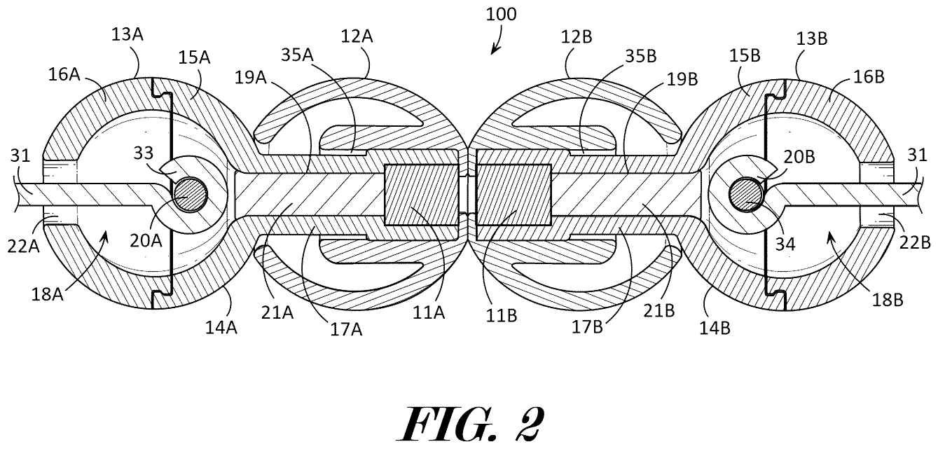

The device 100 may comprise a first plug base 13A and a second plug base 13B that may be removably coupled together via two or more connectors 11A, 11B. In some embodiments, the first plug base 13A may comprise or be coupled to a first connector 11A, and the second plug base 13B may comprise or be coupled to a second connector 11B. In some embodiments, a first connector 11A may be coupled to a first ear plug 12A, such as by being directly coupled to the first ear plug 12A and/or by being coupled to a first plug base 13A that the first ear plug 12A is coupled to, and the first ear plug 12A may surround at least a portion of the first connector 11A. In some embodiments, a second connector 11B may be coupled to a second ear plug 12B, such as by being directly coupled to the second ear plug 12B and/or by being coupled to a second plug base 13B that the second ear plug 12B is coupled to, and the second ear plug 12B may surround at least a portion of the second connector 11B. In preferred embodiments, the first connector 11A may be positioned within the first ear plug 12A so that the first ear plug 12A surrounds the first connector 11A as perhaps best shown in FIGS. 1 and 2, and/or the second connector 11B may be positioned within the second ear plug 12B so that the second ear plug 12B surrounds the second connector 11B as perhaps best shown in FIGS. 1 and 2. Preferably, a connector 11A, 11B, positioned within an ear plug 12A, 12B, may prevent the connector 11A, 11B, from contacting the wearer's ear during normal use of the device 100. Preferably, a first connector 11A may be configured to be removably coupled to a second connector 11B in a manner to enable one-handed operation to facilitate quick and efficient use.

In some embodiments, the device 100 may comprise connectors 11A, 11B, that may be configured to be magnetically coupled together. Magnetic connectors 11A, 11B, may be removably coupled together via magnetic attraction or magnetic connection by having the connectors 11A, 11B, comprise a magnetic material. Example, magnetic materials may include: ferrite, manganese-zinc ferrite, nickel-zinc ferrite, strontium ferrite, cobalt ferrite, barium ferrite, magnetic alloys such as alnico, comol, Hypernom® magnetic alloy, manganese-zinc ferrite, iron-silicon magnet alloys, nickel-zinc ferrite, ferritic stainless steel alloys, strontium ferrite, barium ferrite, alnico, iron-silicon magnet alloy, Chromindur® (Chromium-Cobalt-Iron) alloys, Silmanal (Silver-Manganese-Aluminium) alloys, Platinax II (platinum-cobalt) alloy, Bismanol (manganese bismuthide) alloy, cobalt-platinum alloys, chromium-manganese antimonide alloy, vectolite (cobalt ferrite), magnadur (sintered barium ferrite), lodex (oxide-coated iron-cobalt particles), awaruite (Ni2Fe to Ni3Fe nickel-iron alloy), wairauite, rare earth magnets such as samarium-cobalt, cesium-cobalt, neodymium-iron-boron, other neodymium magnet materials, metallic oxides such as magnetite, ulvospinel, hematite, ilmenite, maghemite, jacobsite, iron sulfides such as pyrrhotite, greigite, troilite, metallic oxyhydroxides such as goethite, lepidocrocite, feroxyhyte, ferrimagnetic materials such as magnetite, pyrrhotite, cubic ferrites, hexagonal ferrites, ferromagnetic materials including metals such as iron, nickel, cobalt, metal alloys containing iron, nickel, and/or cobalt, soft magnetic materials, hard magnetic materials, or any other suitable magnetic material, that is capable of magnetically adhering to another magnetic material through the principle of magnetism.

In preferred embodiments, the device 100 may comprise a first magnet connector 11A that may be coupled to a first plug base 13A and a second magnet connector 11B that may be coupled to a second plug base 13B. A first ear plug 12A may be coupled to the first plug base 13A, and the first ear plug 12A may fully cover the first magnet connector 11A so that the first magnet connector 11A does not come into contact with a user's ear when the first ear plug 12A and first magnet connector 11A are inserted into the user's ear. Likewise, a second ear plug 12B may be coupled to the second plug base 13B, and the second ear plug 12B may fully cover the second magnet connector 11B so that the second magnet connector 11B does not come into contact with a user's ear when the second ear plug 12B and second magnet connector 11B are inserted into the user's ear. Optionally, an ear plug 12A, 12B, may partially cover a magnet connector 11A, 11B, so that the distal end of the magnet connector 11A, 11B, (end distal to flexible coupling 31) is exposed in or extends out of an ear plug aperture 36A, 36B, of an ear plug 12A, 12B. In alternative embodiments, magnetic connectors 11A, 11B, may be coupled to any other part of their respective plug bases 13A, 13B.

In further embodiments, a flexible coupling 31, a coupling retainer 20A, 20B and/or an acoustic dampener 21A, 21B may comprise a sound dampening material, which may include elastomers, visco-elastic polyurethane materials, rubber, cork, foam materials, laminate materials, and the like. Elastomers may include unsaturated rubbers that can be cured by sulfur vulcanization, such as Natural polyisoprene: cis-1,4-polyisoprene natural rubber (NR) and trans-1,4-polyisoprene gutta-percha; Synthetic polyisoprene (IR for isoprene rubber); Polybutadiene (BR for butadiene rubber); Chloroprene rubber (CR), polychloroprene, Neoprene, Baypren etc.; Butyl rubber (copolymer of isobutylene and isoprene, IIR); Halogenated butyl rubbers (chloro butyl rubber: CIIR; bromo butyl rubber: BIIR); Styrene-butadiene Rubber (copolymer of styrene and butadiene, SBR); Nitrile rubber (copolymer of butadiene and acrylonitrile, NBR), also called Buna N rubbers; Hydrogenated Nitrile Rubbers (HNBR) Therban and Zetpol; and the like (Unsaturated rubbers can also be cured by non-sulfur vulcanization if desired), and Elastomers may also include Saturated rubbers that cannot be cured by sulfur vulcanization: EPM (ethylene propylene rubber, a copolymer of ethylene and propylene) and EPDM rubber (ethylene propylene diene rubber, a terpolymer of ethylene, propylene and adiene-component); Epichlorohydrin rubber (ECO); Polyacrylic rubber (ACM, ABR); Silicone rubber (SI, Q, VMQ); Fluorosilicone Rubber (FVMQ); Fluoroelastomers (FKM, and FEPM) Viton, Tecnoflon, Fluorel, Aflas and Dai-El; Perfluoroelastomers (FFKM) Tecnoflon PFR, Kalrez, Chemraz, Perlast; Polyether block amides (PEBA); Chlorosulfonated polyethylene (CSM), (Hypalon); Ethylene-vinyl acetate (EVA); and the like. Foam materials may include silicone foams, rubber foams, urethane foams including ARTiLAGE foams and Poron foams, plastic foams, neoprene foam, latex foam rubber, polyurethane foam rubber, such as polyether polyurethane foam and polyester polyurethane foam, or any other similar material.

While some exemplary shapes and sizes have been provided for elements of the device 100, it should be understood to one of ordinary skill in the art that the connectors 11A, 11B, ear plugs 12A, 12B, plug bases 13A, 13B, flexible coupling 31, and any other element described herein may be configured in a plurality of sizes and shapes including “T” shaped, “X” shaped, square shaped, rectangular shaped, cylinder shaped, cuboid shaped, hexagonal prism shaped, triangular prism shaped, or any other geometric or non-geometric shape, including combinations of shapes. It is not intended herein to mention all the possible alternatives, equivalent forms or ramifications of the invention. It is understood that the terms and proposed shapes used herein are merely descriptive, rather than limiting, and that various changes, such as to size and shape, may be made without departing from the spirit or scope of the invention.

Additionally, while some materials have been provided, in other embodiments, the elements that comprise the device 100 may be made from or may comprise durable materials such as aluminum, steel, other metals and metal alloys, wood, hard rubbers, hard plastics, fiber reinforced plastics, carbon fiber, fiberglass, resins, polymers or any other suitable materials including combinations of materials. Additionally, one or more elements may be made from or may comprise durable and slightly flexible materials such as soft plastics, silicone, soft rubbers, or any other suitable materials including combinations of materials. In some embodiments, one or more of the elements that comprise the device 100 may be coupled or connected together with heat bonding, chemical bonding, adhesives, clasp type fasteners, clip type fasteners, rivet type fasteners, threaded type fasteners, other types of fasteners, or any other suitable joining method. In other embodiments, one or more of the elements that comprise the device 100 may be coupled or removably connected by being press fit or snap fit together, by one or more fasteners such as hook and loop type or Velcro® fasteners, magnetic type fasteners, threaded type fasteners, sealable tongue and groove fasteners, snap fasteners, clip type fasteners, clasp type fasteners, ratchet type fasteners, a push-to-lock type connection method, a turn-to-lock type connection method, a slide-to-lock type connection method or any other suitable temporary connection method as one reasonably skilled in the art could envision to serve the same function. In further embodiments, one or more of the elements that comprise the device 100 may be coupled by being one of connected to and integrally formed with another element of the device 100.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A wearable noise-reducing device, the device comprising:
a first connector coupled to a first ear plug, the first ear plug surrounding at least a portion of the first connector;
a second connector coupled to a second ear plug, the second ear plug surrounding at least a portion of the second connector, wherein the first connector and second connector are configured to be removably coupled together; and
a flexible coupling having an elongated length, wherein the flexible coupling is coupled to both the first ear plug and to the second ear plug.

2. The device of claim 1, further comprising a first plug base, and wherein the first connector, the first ear plug, and the flexible coupling are coupled to the first plug base.

3. The device of claim 1, wherein the first connector is positioned within the first ear plug so that the first ear plug surrounds the first connector.

4. The device of claim 3, wherein the second connector is positioned within the second ear plug so that the second ear plug surrounds the second connector.

5. The device of claim 1, wherein the first ear plug and the second ear plug each comprise a flexible material.

6. The device of claim 1, wherein the first connector comprises a first magnetic material, wherein the second connector comprises a second magnetic material, and wherein the first connector and the second connector configured to be magnetically coupled together.

7. The device of claim 1, wherein the first plug base comprises a base projection, wherein the first ear plug and the first connector are coupled to the base projection, and wherein the base projection comprises an acoustic dampener.

8. The device of claim 7, wherein the first plug base comprises a base cavity, wherein the base projection comprises a base channel that extends through the base projection and into communication with the base cavity, and wherein the acoustic dampener is disposed within the base channel to acoustically isolate the first connector from the base cavity.

9. The device of claim 7, wherein the acoustic dampener comprises acoustic foam.

10. The device of claim 1, wherein the first plug base comprises a base cavity, wherein the flexible coupling comprises a first end, and wherein the first end of the flexible coupling is coupled to the plug base within the base cavity.

11. The device of claim 10, wherein the first end of the flexible coupling is coupled to a coupling retainer that is positioned within the base cavity.

12. A wearable noise-reducing device, the device comprising:
a first connector coupled to a first plug base;
a first ear plug coupled to the first plug base, the first ear plug surrounding at least a portion of the first connector;
a second connector coupled to a second plug base, wherein the first connector and second connector are configured to be removably coupled together;
a second ear plug coupled to the second plug base, the second ear plug surrounding at least a portion of the second connector; and
a flexible coupling having an elongated length, wherein the flexible coupling is coupled to both the first plug base and to the second plug base.

13. The device of claim 12, wherein the first connector is positioned within the first ear plug so that the first ear plug surrounds the first connector.

14. The device of claim 13, wherein the second connector is positioned within the second ear plug so that the second ear plug surrounds the second connector.

15. The device of claim 12, wherein the first ear plug and the second ear plug each comprise a flexible material.

16. The device of claim 12, wherein the first connector comprises a first magnetic material, wherein the second connector comprises a second magnetic material, and wherein the first connector and the second connector configured to be magnetically coupled together.

17. The device of claim 12, wherein the first plug base comprises a base projection, wherein the first ear plug and the first connector are coupled to the base projection, and wherein the base projection comprises an acoustic dampener.

18. The device of 17, wherein the acoustic dampener comprises acoustic foam.

19. The device of claim 12, wherein the first plug base comprises a base cavity, wherein the flexible coupling comprises a first end, and wherein the first end of the flexible coupling is coupled to the plug base within the base cavity.

20. The device of claim 19, wherein the first end of the flexible coupling is coupled to a coupling retainer that is positioned within the base cavity.
